(19) United States
(12) Patent Application Publication
(10) Pub. No.: US 2026/0191646 A1
(43) Pub. Date: Jul. 9, 2026

(54) POSITIONING MEMBER FOR A PROSTHETIC DEVICE DELIVERY APPARATUS

(71) Applicant: Edwards Lifesciences Corporation, Irvine, CA (US)

(72) Inventors: Jeong Soo Lee, Yorba Linda, CA (US); Grant Jason Gangeness, Carlsbad, CA (US)

(21) Appl. No.: 19/551,515

(22) Filed: Feb. 26, 2026

Related U.S. Application Data
(63) Continuation of application No. PCT/US2024/044918, filed on Aug. 31, 2024.
(60) Provisional application No. 63/580,306, filed on Sep. 1, 2023.

(51) Int. Cl. A61F 2/24

(52) U.S. Cl. CPC A61F 2/2433; A61F 2/2418; A61F 2230/0006; A61F 2250/0097; A61F 2250/0098

(57) ABSTRACT

A delivery apparatus including a positioning member for axially aligning a prosthetic valve onto the delivery apparatus is disclosed. The delivery apparatus can include a shaft defining a lumen for a guidewire, where the shaft is a first color, an inflatable balloon overlaying a distal end portion of the shaft, where the balloon is configured to receive a prosthetic valve in a radially compressed state, a radiopaque marker mounted to the distal end portion of the shaft, and a polymeric positioning member disposed around the distal end portion of the shaft and inside the balloon. The positioning member is a second color, and the positioning member is configured to indicate an axial position for an end of the prosthetic valve when the prosthetic valve is mounted around the balloon.

### CROSS-REFERENCE TO RELATED APPLICATIONS

This application is a continuation of PCT Patent Application No. PCT/US2024/044918 filed on August 31, 2024, which application claims the benefit of U.S. Provisional Patent Application No. 63/580,306, filed on September 1, 2023, each of these applications being incorporated by reference herein in its entirety.

### FIELD

The present disclosure relates to prosthetic heart valves, delivery apparatuses for prosthetic heart valves, and systems and methods for mounting a prosthetic heart valve onto a valve mounting portion of a delivery apparatus.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

### SUMMARY

Described herein are delivery apparatuses, valve retention members, positioning members, and methods for positioning and radially compressing a prosthetic heart valve onto a distal end portion of a delivery apparatus. The prosthetic heart valves delivered with the delivery apparatuses disclosed herein are, for example, radially expandable from a radially compressed state mounted on the delivery apparatus to a radially expanded state for implantation using an inflatable balloon (or an equivalent expansion device) of the delivery apparatus. Although the devices and methods disclosed herein are particularly suited for delivery apparatuses for prosthetic heart valves (for example, a prosthetic aortic valve or prosthetic mitral valve), the disclosed devices and methods can be adapted for positioning and delivering other types of prosthetic valves (for example, prosthetic venous valves) or other types of expandable prosthetic devices adapted to be implanted in various body lumens.

The disclosed prosthetic heart valves, delivery apparatuses, and methods can, for example, enable a prosthetic heart valve to be mounted onto a valve mounting portion of a delivery apparatus in a specified position (e.g., axial position relative to a portion of the delivery apparatus, such as a distal shoulder and/or nose cone). As such, the devices and methods disclosed herein can, among other things, overcome one or more of the deficiencies of typical prosthetic heart valves and their delivery apparatus.

A delivery apparatus for a prosthetic implant can comprise a handle and one or more shafts coupled to the handle.

A delivery apparatus for a prosthetic implant can comprise a shaft defining a lumen for a guidewire and an inflatable balloon overlaying a distal end portion of the shaft, wherein the balloon is configured to receive a prosthetic valve in a radially compressed state.

In some examples, the delivery apparatus can comprise a handle and the shaft can be coupled to the handle.

In some examples, the delivery apparatus can comprise a positioning member disposed around a distal end portion of the shaft and inside the balloon.

In some examples, the shaft is a first color, and the positioning member can comprise a polymer and has second color.

In some examples, the delivery apparatus can comprise a radiopaque marker mounted to the distal end portion of the shaft.

In some examples, the positioning member is disposed around the radiopaque marker.

In some examples, the delivery apparatus can comprise a distal shoulder, and the positioning member is molded as one piece with the distal shoulder, and where the positioning member extends past and proximal to a proximal end of a flared portion of the distal shoulder.

In some examples, a delivery apparatus comprises a shaft defining a lumen for a guidewire, wherein the shaft is a first color; an inflatable balloon overlaying a distal end portion of the shaft, wherein the balloon is configured to receive a prosthetic valve in a radially compressed state; a radiopaque marker mounted to the distal end portion of the shaft; and a polymeric positioning member disposed around the distal end portion of the shaft and inside the balloon, wherein the positioning member is a second color, and wherein the positioning member is configured to indicate an axial position for an end of the prosthetic valve when the prosthetic valve is mounted around the balloon.

In some examples, a delivery apparatus comprises a shaft defining a lumen for a guidewire; an inflatable balloon arranged around a distal end portion of the shaft and overlaying a valve mounting portion of the delivery apparatus that is configured to receive a prosthetic valve in a radially compressed state; and a distal shoulder mounted on the distal end portion of the shaft and arranged within a distal end portion of the balloon, wherein the distal shoulder comprises a base portion, a flared portion that extends radially outward from the base portion and proximally toward the valve mounting portion, and a positioning member extending axially from the base portion and proximal to a proximal end of the flared portion, wherein the positioning member is formed as one piece with a remainder of the distal shoulder.

In some examples, a delivery apparatus comprises one or more of the components recited in Examples 1-15, 17-31, and 43-52 below.

A valve retention member for a delivery apparatus for a prosthetic valve can comprise a base portion, a flared portion that extends radially outward and proximally from the base portion, and a positioning member that extends proximally from the base portion and past a proximal end of the flared portion.

In some examples, the positioning member, flared portion, and base portion are formed or molded together as a unitary component.

In some examples, the base portion configured to be mounted to a distal end portion of a shaft of the delivery apparatus.

In some examples, the positioning member has a distinguishing indicium visible to a user to aid in positioning of the prosthetic valve onto the delivery apparatus during crimping of the prosthetic valve to the delivery apparatus.

In some examples, the indicium is a first color that is distinguishable to the user under visible light.

In some examples, the indicium is a texture or raised feature.

In some examples, the indicium is a visible shape or symbol on a base structure of the positioning member.

In some examples, a valve retention member for a delivery apparatus for a prosthetic valve comprises a base portion configured to be mounted to a distal end portion of a shaft of the delivery apparatus; a flared portion that extends radially outward and proximally from the base portion, wherein a proximal direction is parallel to a central longitudinal axis of the valve retention member; and a positioning member that extends proximally from the base portion and past a proximal end of the flared portion, wherein the positioning member has a distinguishing indicium visible to a user to aid in positioning of the prosthetic valve onto the delivery apparatus during crimping of the prosthetic valve to the delivery apparatus, and wherein the positioning member, flared portion, and base portion are formed together as a unitary component.

In some examples, a valve retention member comprises one or more of the components recited in Examples 32-41 below.

An assembly can comprise a prosthetic heart valve and a delivery apparatus.

In some examples, the delivery apparatus can comprise a shaft defining a lumen for a guidewire, an inflatable balloon overlaying a distal end portion of the shaft, and a positioning member disposed around the distal end portion of the shaft and inside the balloon.

In some examples, the prosthetic heart valve is radially collapsible to a collapsed configuration and radially expandable to an expanded configuration, the prosthetic valve mounted around the balloon on the shaft in the collapsed configured with its distal end axially aligned with a proximal end of the positioning member.

In some examples, an assembly comprises one or more of the components recited in Examples 16, 42, and 53-55 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of a prosthetic heart valve, according to an example.

FIG. 1B is a perspective view of the prosthetic valve of FIG. 1A with the components on the outside of the frame shown in transparent lines for purpose of illustration.

FIG. 2 is a side view of an exemplary delivery apparatus configured to deliver and implant a radially expandable prosthetic heart valve at an implantation site.

FIG. 3 is a cross-sectional side view of a distal end portion of the delivery apparatus of FIG. 2.

FIG. 4 is a side view of the distal end portion of the delivery apparatus of FIG. 2.

FIG. 5A is a side view of the distal end portion of the delivery apparatus of FIG. 3, which illustrates a positioning member disposed around an inner shaft of the delivery apparatus and extending proximally from a distal shoulder of the delivery apparatus.

FIG. 5B is a side view of the distal end portion of the delivery apparatus of FIG. 5A, which illustrates a prosthetic valve mounted on a valve mounting portion of the delivery apparatus with its distal end aligned with a proximal end of the positioning member.

FIG. 5C is a side view of the distal end portion of the delivery apparatus of FIG. 3, which illustrates a prosthetic valve mounted on a valve mounting portion of the delivery apparatus with its distal end aligned with a proximal end of a first positioning member disposed around an inner shaft of the delivery apparatus and its proximal end aligned with a distal end of a second positioning member disposed around the inner shaft.

FIG. 6 is a cross-sectional side view of an integrated distal shoulder and positioning member for the delivery apparatus of FIG. 2.

FIG. 7 is a perspective view of an assembly including the integrated distal shoulder and positioning member of FIG. 6 and a nose cone.

FIG. 8 is a cross-sectional side view of a distal shoulder and a separate positioning member configured to be assembled onto a distal end of a delivery apparatus.

FIG. 9 is a perspective view of a prosthetic heart valve, according to an example.

FIG. 10 is a perspective view of a prosthetic heart valve, according to an example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient’s body), while distal motion of the device is motion of the device away from the user and toward the implantation site (e.g., into the patient’s body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

As introduced above, prosthetic heart valves can be mounted onto a delivery apparatus in a crimped or radially compressed state for delivery to a target implantation site using the delivery apparatus. The delivery apparatus can include an inflatable balloon overlaying a valve mounting portion that is configured to receive the prosthetic heart valve. However, in some instances, there may be some freedom in mounting the prosthetic valve onto the valve mounting portion in various axial positions (e.g., due to the valve mounting portion being longer than a length of the radially compressed prosthetic valve). A distal end of the valve mounting portion can be defined by a distal shoulder (or valve retention member) of the delivery apparatus. A distance of an end of the radially compressed prosthetic valve on the valve mounting portion from the distal shoulder of the delivery apparatus can affect a deployment shape of the prosthetic valve as it is radially expanded (by inflating the balloon) at the implantation site. For example, if the prosthetic valve is mounted too close to the distal shoulder, upon inflation of the balloon, the prosthetic valve deploys less symmetrically (e.g., one end expanding faster than the other end). This may cause inaccuracies in implanting the prosthetic valve at the implantation site. Thus, there may be an optimal distance or distance range between the radially compressed prosthetic valve and distal shoulder of the delivery apparatus that results in more even deployment upon inflation of the balloon. The optimal distance or distance range can also be based on (and change for) the target anatomy for implantation of the prosthetic valve, such as the aortic annulus or mitral annulus. By mounting the radially compressed prosthetic valve onto the delivery apparatus at the optimal distance or distance rage from the distal shoulder, the prosthetic valve can be implanted in a more consistent and accurate manner.

In order to mount the prosthetic valve onto the valve mounting portion in a specified axial position relative to a portion of the delivery apparatus (e.g., the distal shoulder) such that it deploys more symmetrically, one or more positioning members or visual aids can be used on the delivery apparatus. In some examples, a positioning member can comprise a visible and distinguishable material (visible under visible light) or other indicum or indicia that extends a distance from the distal shoulder along the valve mounting portion. An end of the positioning member that is disposed away from the distal shoulder can indicate a specified location for the distal end of the prosthetic heart valve to be positioned on the valve mounting portion (in an at least partially radially compressed configuration). In some instances, the positioning member can be a tube positioned around an inner shaft of the delivery apparatus and extending proximally away from the distal shoulder. In some instances, the positioning member can be part of the distal shoulder (formed as one piece).

In some examples, the delivery apparatus can include two positioning members spaced axially apart from one another, where a space between the two positioning members is configured to receive the radially compressed prosthetic valve. In this way, a first, distal positioning member of the two positioning members can indicate a location for a distal end for the prosthetic valve and a second, proximal positioning member of the two positioning members can indicate a location for a proximal end for the prosthetic valve.

The positioning member can be easily and highly visible under ambient light (or visible light). In some instances, the positioning member can have a contrasting color from the inner shaft (or guidewire lumen) of the delivery apparatus, where the contrasting color is easily and highly visible under visible or ambient light. For example, in some instances, the positioning member can comprise a polymer having a different color than the inner shaft of the delivery apparatus, where the different color is easily visible under ambient light of a procedure or operating room. In this way, the positioning member (or members) can be easily visualized and distinguished from the rest of the delivery apparatus in a procedure room, thereby allowing a user to mount the prosthetic valve easily and quickly onto the valve mounting portion of the delivery apparatus, at the specified distance from the distal shoulder and/or between two positioning members. As a result, the prosthetic valve can be radially compressed onto the delivery apparatus in a target axial position along the valve mounting portion that results in more symmetric deployment of the prosthetic valve at the implantation site.

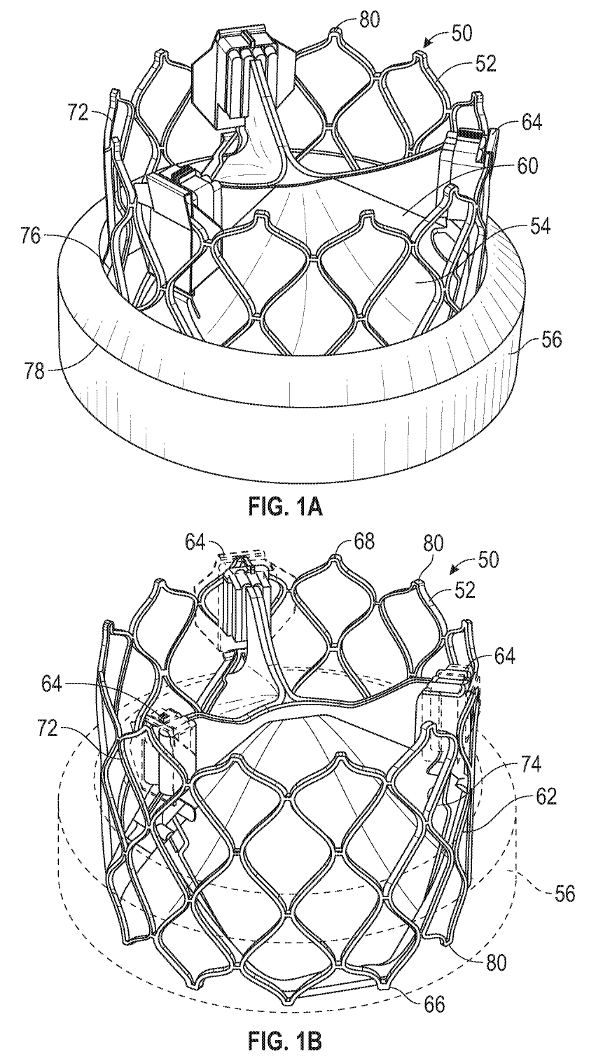

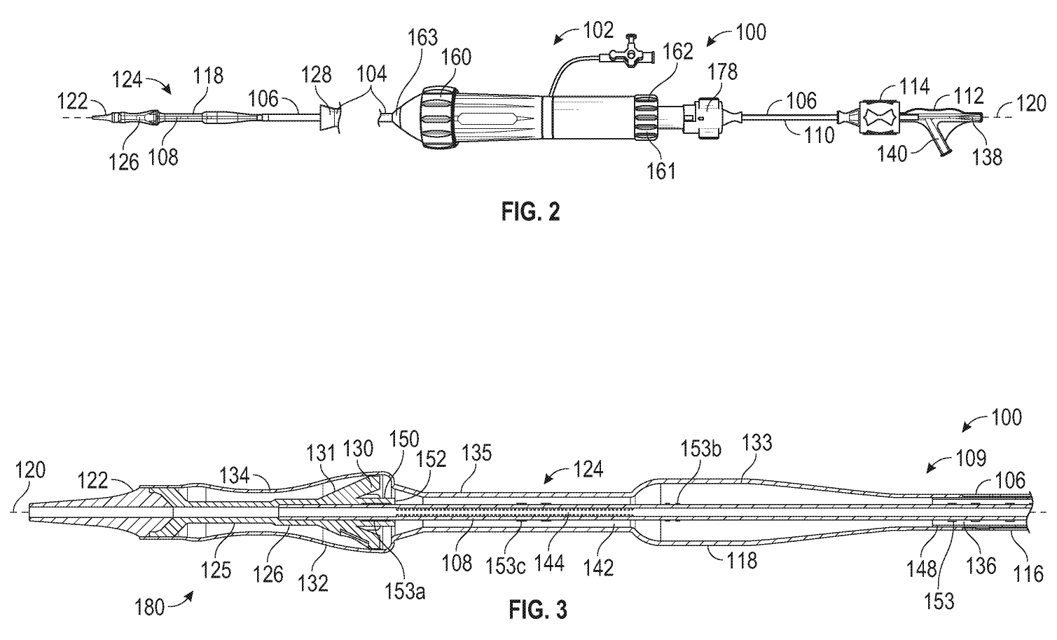

FIGS. 1A and 1B show an exemplary prosthetic heart valve in a radially expanded configuration that can be crimped into a radially compressed configuration onto a delivery apparatus (e.g., the delivery apparatus of FIGS. 2). In particular, the delivery apparatus can comprise a valve mounting portion at its distal end portion, covered by an inflatable balloon, that is configured to receive the radially compressed prosthetic valve thereon, as shown in FIGS. 3-5C).

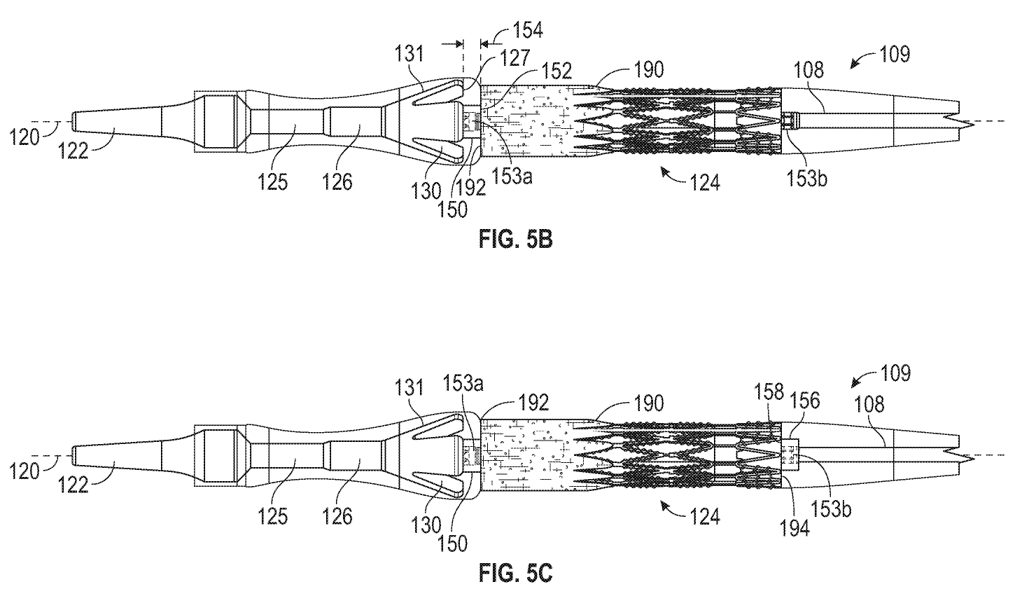

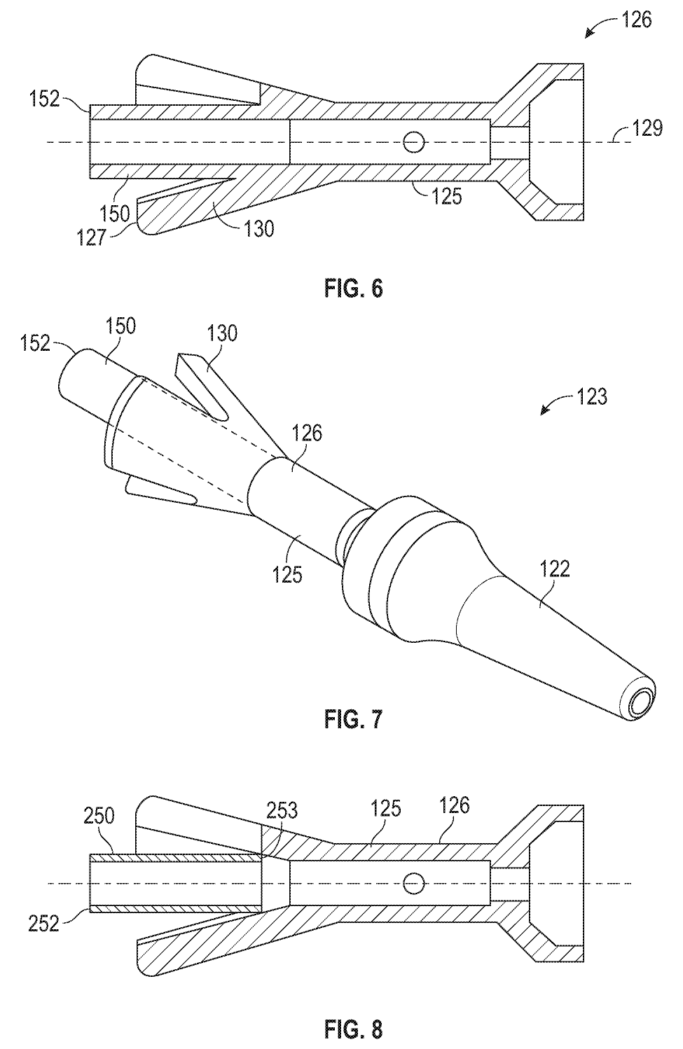

FIGS. 3 and 5A-5C depict an exemplary positioning member disposed around an inner shaft (which forms a guidewire lumen) and extending along a specified portion of the valve mounting portion. For example, in some instances, the positioning member can extend proximally from a distal shoulder of the delivery apparatus, where a proximal end of the positioning member indicates an end position for a distal end of the prosthetic valve to be mounted on the valve mounting portion (FIGS. 5A and 5B). In some examples, the delivery apparatus can include an additional positioning member at a proximal end of the valve mounting portion that indicates an end position for a proximal end of the prosthetic valve to be mounted on the valve mounting portion (FIG. 5C). In some instances, the distal positioning member can be formed as part of the distal shoulder the delivery apparatus (FIGS. 6 and 7).

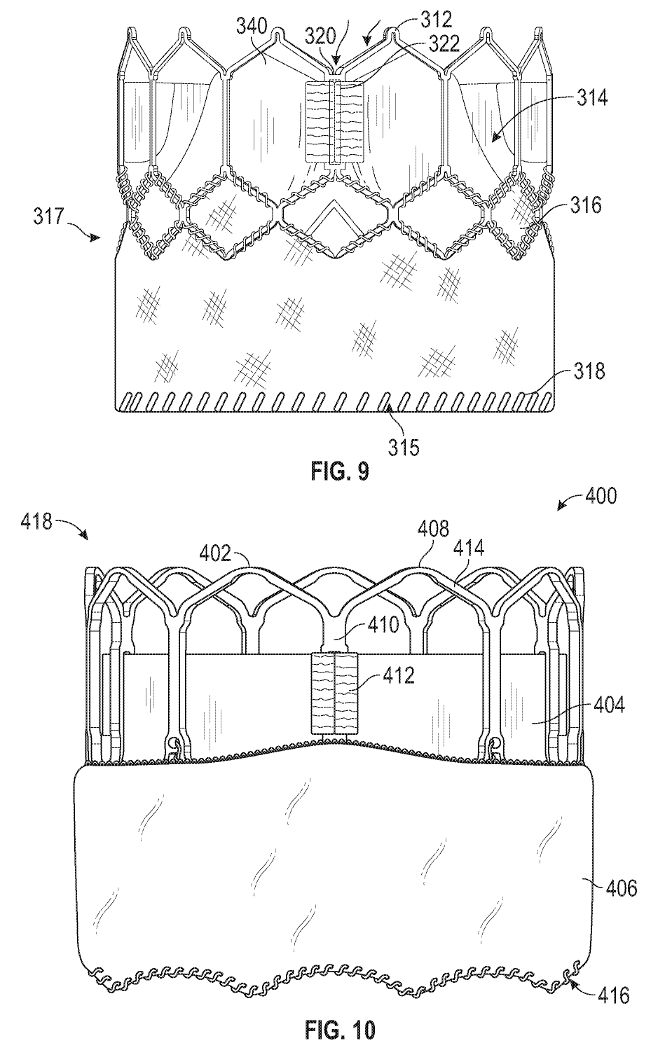

FIGS. 8 and 9 show additional examples of prosthetic heart valves that can be used with the delivery apparatuses described herein.

### Examples of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient’s vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

FIGS. 1A and 1B show an exemplary prosthetic valve 50, according to an example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 50 can have three main components: a stent or frame, 52, a valvular structure 54, and a sealing member 56 (FIG. 1A). FIG. 1B is a perspective view of the prosthetic valve 50 with the components on the outside of the frame 52 (including the sealing member 56) shown in transparent lines for purposes of illustration. The prosthetic valve 50 can have an inflow end 66 and an outflow end 68.

The valvular structure 54 can comprise three leaflets 60, collectively forming a leaflet structure, which can be arranged to collapse in a tricuspid arrangement, although in other examples there can be greater or fewer number of leaflets (e.g., one or more leaflets 60). In some examples, the leaflets 60 can be formed of pericardial tissue (e.g., bovine pericardial tissue), biocompatible synthetic materials, or various other suitable natural or synthetic materials as known in the art and described in U.S. Patent No. 6,730,118, which is incorporated by reference herein.

Each leaflet 60 can be coupled to the frame 52 along its inflow edge 62 (the lower edge in the figures; also referred to as “cusp edges”) and at commissures 64 of the valvular structure 54 where adjacent portions (e.g., commissure tabs) of two leaflets are connected to each other. In some examples, the commissures 64 can comprise an attachment member (e.g., comprising fabric, flexible polymer, or the like) arranged across a cell (e.g., commissure cell) of the frame 52, the cell formed by struts of the frame. The attachment member can be secured to the struts of the frame forming the cell and the adjacent portions of the two leaflets can be connected to the attachment member to form the commissure 64 (e.g., as shown in FIGS. 3-8, as described further below).

In some examples, a reinforcing element or connecting skirt, such as a fabric strip, can be connected directly to the cusp edges of the leaflets and to the struts of the frame to couple the cusp edges of the leaflets to the frame.

The frame 52 can be made of any of various suitable plastically-expandable materials (e.g., stainless steel, etc.) or self-expanding materials (e.g., Nitinol). When constructed of a plastically-expandable material, the frame 52 (and thus the prosthetic valve 50) can be crimped to a radially collapsed configuration on a delivery apparatus (e.g., catheter) and then expanded inside a patient by an inflatable balloon or equivalent expansion mechanism. Various crimping devices can be used to crimp the prosthetic valve 50 and the other prosthetic valves described herein around the delivery apparatus, such as the crimping devices described in U.S. Patent No. 7,530,253, which is incorporated herein by reference.

In some instances, the prosthetic valve 50 can be crimped directly onto the inflatable balloon of the delivery apparatus, such that the prosthetic valve 50 is axially aligned with and disposed radially outward of the balloon during advancing the prosthetic valve on the delivery apparatus to the implantation site, for example as described in PCT Application No. PCT/US2021/047056, which is incorporated herein by reference. In alternate instances, the prosthetic valve 50 can be crimped onto the delivery apparatus axially offset from the balloon, and then moved over the balloon at the implantation site, prior to inflation of the balloon and radial expansion of the prosthetic valve, such as described in U.S. Patent Application 9,339,384, which is incorporated herein by reference.

When constructed of a self-expandable material, the frame 52 (and thus the prosthetic valve 50) can be crimped to a radially collapsed configuration and restrained in the collapsed configuration by insertion into a sheath or equivalent mechanism of a delivery apparatus. Once inside the body, the prosthetic valve can be advanced from the delivery sheath, which allows the prosthetic valve to expand to its functional size.

Suitable plastically-expandable materials that can be used to form the frame 52 include, metal alloys, polymers, or combinations thereof. Example metal alloys can comprise one or more of the following: nickel, cobalt, chromium, molybdenum, titanium, or other biocompatible metal. In some examples, the frame 52 can comprise stainless steel. In some examples, the frame 52 can comprise cobalt-chromium. In some examples, the frame 52 can comprise nickel-cobalt-chromium. In some examples, the frame 52 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

The frame 52 in the illustrated example comprises a plurality of circumferentially extending rows of angled struts 72 defining rows of open cells 74 (or openings) of the frame. The frame 52 can have a cylindrical or substantially cylindrical shape having a constant diameter from the inflow end 66 to the outflow end 68 of the frame 52 as shown, or the frame 52 can vary in diameter along the height of the frame, as disclosed in U.S. Patent Publication No. 2012/0239142, which is incorporated herein by reference.

The frame 52, at each of the inflow end 66 and the outflow end 68, may comprise a plurality of apices 80 spaced apart from one another around a circumference of the frame 52.

The sealing member 56 in the illustrated example is mounted on the outside of the frame 52 and functions to create a seal against the surrounding tissue (e.g., the native leaflets and/or native annulus) to prevent or at least minimize paravalvular leakage. The sealing member 56 can comprise an inner layer 76 (which can be in contact with the outer surface of the frame 52) and an outer layer 78. The sealing member 56 can be connected to the frame 52 using suitable techniques or mechanisms. For example, the sealing member 56 can be sutured to the frame 52 via sutures that can extend around the struts 72 and through the inner layer 76. In alternative examples, the inner layer 76 can be mounted on the inner surface of the frame 52, while the outer layer 78 is on the outside of the frame 52.

The outer layer 78 can be configured or shaped to extend radially outward from the inner layer 76 and the frame 52 when the prosthetic valve 50 is deployed. When the prosthetic valve is fully expanded outside of a patient’s body, the outer layer 78 can expand away from the inner layer 76 to create a space between the two layers. Thus, when implanted inside the body, this allows the outer layer 78 to expand into contact with the surrounding tissue.

Additional details regarding the prosthetic valve 50 and its various components are described in U.S. Patent Publication No. 2018/0028310, which is incorporated herein by reference.

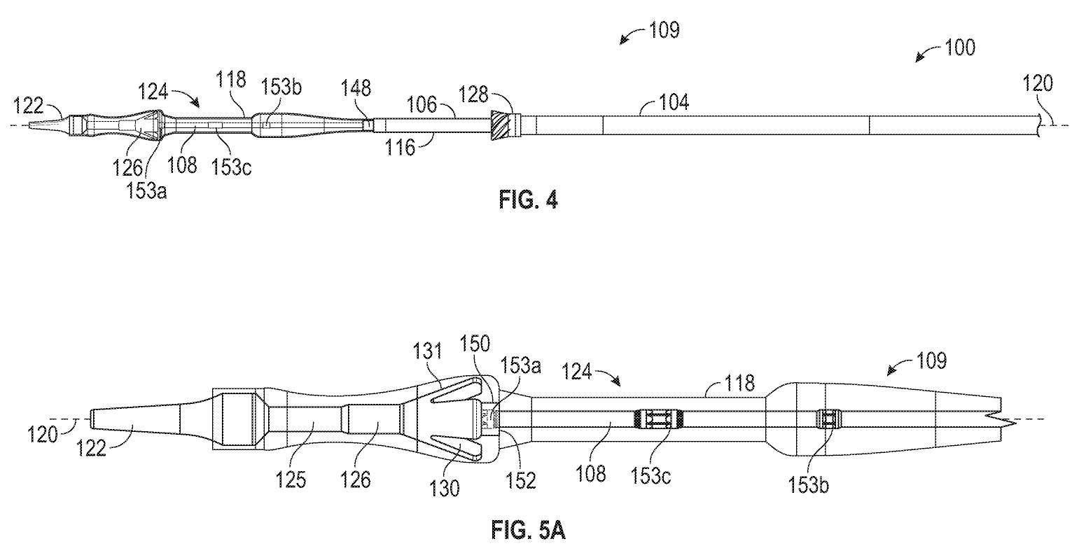

FIG. 2 shows an example delivery apparatus 100, which can be used to implant an expandable prosthetic heart valve (for example, the prosthetic valve 50 of FIG. 1A), or another type of expandable prosthetic medical device (such as a stent). A distal end portion 109 of the delivery apparatus 100 is shown in FIGS. 3 and 4 (a cross-sectional view in FIG. 3 and a side view in FIG. 4). In some examples, the delivery apparatus 100 is specifically adapted for use in introducing a prosthetic valve into a heart. As described further below, the delivery apparatus 100 can be configured to rotate the prosthetic valve, mounted on the delivery apparatus in a radially compressed state, at the target implantation site (for example, at a native valve of the heart) to achieve commissure alignment between the native valve and prosthetic valve after deploying the prosthetic valve.

In the depicted example, the delivery apparatus 100 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends both proximally and distally from the handle 102. The portion of the intermediate shaft 106 extending distally from the handle 102 also extends coaxially through the outer shaft 104. Additionally, the delivery apparatus 100 can further comprise an inner shaft 108 extending distally from the handle 102 and coaxially through the intermediate shaft 106 and the outer shaft 104. The inner shaft 108 also extends proximally from the handle 102 and coaxially through the intermediate shaft 106.

The outer shaft 104 and the intermediate shaft 106 are configured to translate longitudinally, along a central longitudinal axis 120 of the delivery apparatus 100, relative to one another to facilitate delivery and positioning of a prosthetic valve at an implantation site in a patient’s body.

The intermediate shaft 106 can include a proximal end portion 110 that extends proximally from a proximal end of the handle 102, to an adaptor 112. A rotatable knob 114 can be mounted on the proximal end portion 110. The knob 114 can be configured to rotate the intermediate shaft 106 around the central longitudinal axis 120 of the delivery apparatus 100 and relative to the outer shaft 104.

The adaptor 112 can include a first port 138 configured to receive a guidewire therethrough and a second port 140 configured to receive fluid (for example, inflation fluid) from a fluid source. The second port 140 can be fluidly coupled to an inner lumen of the intermediate shaft 106.

The intermediate shaft 106 can further include a distal end portion 116 that extends distally beyond a distal end of the outer shaft 104 when the distal end of the outer shaft 104 is positioned away from an inflatable balloon 118 of the delivery apparatus (FIGS. 3 and 4). A distal end portion of the inner shaft 108 can extend distally beyond the distal end portion 116 of the intermediate shaft 106.

The balloon 118 can be coupled to the distal end portion 116 of the intermediate shaft 106. For example, a proximal end portion of the balloon 118 can be coupled to and/or around a distal end 148 of the intermediate shaft 106 (FIG. 3).

The balloon 118 can comprise a distal end portion (or section) 132, a proximal end portion (or section) 133, and an intermediate portion (or section) 135, the intermediate portion 135 disposed between the distal end portion 132 and the proximal end portion 133 (FIG. 3).

In some examples, a distal end of the distal end portion 132 of the balloon 118 can be coupled to a distal end of the delivery apparatus 100, such as to a nose cone 122, or to an alternate component at the distal end of the delivery apparatus 100 (for example, a distal shoulder). In some examples, the intermediate portion 135 of the balloon 118 can overlay a valve mounting portion 124 of a distal end portion 109 of the delivery apparatus 100, the distal end portion 132 can overly a distal shoulder 126 of the delivery apparatus 100, and the proximal end portion 133 can surround a portion of the inner shaft 108 (FIG. 3). The valve mounting portion 124 and the intermediate portion 135 of the balloon 118 can be configured to receive a prosthetic valve in a radially compressed state (as shown in FIGS. 5B and 5C, which are described further below).

Rotation of the intermediate shaft 106 can cause rotation of the balloon 118 and the prosthetic valve mounted thereon for rotational positioning of the prosthetic valve relative to the native anatomy at the target implantation site.

The delivery apparatus 100 can include a balloon shoulder assembly 180 configured to maintain the prosthetic heart valve or other medical device at a fixed position on the balloon 118 during delivery through the patient’s vasculature (FIG. 3). The balloon shoulder assembly 180 can include a distal shoulder 126 arranged within a distal end portion of the balloon 118 and coupled to the distal end portion of the inner shaft 108. The distal shoulder 126 can be configured to resist movement of the prosthetic valve or other medical device mounted on the valve mounting portion 124 distally, in an axial direction (for example, along the central longitudinal axis 120), relative to the balloon 118.

In some instances, the distal shoulder 126 can be referred to as a valve retention member.

For example, the distal shoulder 126 can include a flared portion 131 arranged adjacent to the valve mounting portion 124. In some examples, the flared portion 131 can include a plurality of wings 130 that flare radially outward from a base portion 125 (for example, shaft) of the distal shoulder 126, toward the valve mounting portion 124.

The outer shaft 104 can include a distal tip portion 128 mounted on its distal end (FIGS. 2 and 4). In some examples, the distal tip portion 128 can be configured as a flex adaptor including a plurality of inner and outer helical grooves. The outer shaft 104 and the intermediate shaft 106 can be translated axially relative to one another to position the distal tip portion 128 adjacent to a proximal end of the valve mounting portion 124, when a prosthetic valve is mounted in the radially compressed state on the valve mounting portion 124 and during delivery of the prosthetic valve to the target implantation site. As such, the distal tip portion 128 can be configured to resist movement of the prosthetic valve relative to the balloon 118 proximally, in the axial direction, relative to the balloon 118, when the distal tip portion 128 is arranged adjacent to a proximal side of the valve mounting portion 124.

In some examples, the nose cone 122 can be disposed distal to and be coupled to the distal shoulder 126 (for example, as shown in FIGS. 5A-5C and FIG. 7, which are described further below).

In some examples, the nose cone 122 can be coupled to the distal end portion of the inner shaft 108.

In some examples, the delivery apparatus 100 can comprise one or more markers or marker bands. For example, the delivery apparatus 100 comprises three marker bands 153a, 153b, and 153c, which are collectively or generically referred to as “the one or more marker bands 153” or “the marker bands 153.” The one or more marker bands 153 are configured to indicate to a user a location of a specified component of the delivery apparatus. In some examples, the one or more marker bands 153 can be radiopaque. For example, the one or more marker bands 153 can comprise a radiopaque material (e.g., platinum iridium) that is visible under medical imaging (such as fluoroscopy). In some examples, one or more marker bands 153 can be radially compressed (for example, crimped) onto the inner shaft 108.

In some examples, the distal end portion 132 of the balloon 118 can include a radial depression 134 that is depressed radially inwardly, toward the central longitudinal axis 120, relative to an outermost radial surface of the distal shoulder 126 and an outermost radial surface of the nose cone 122.

An annular space 136 can be defined between an outer surface of the inner shaft 108 and an inner surface of the intermediate shaft 106 (FIG. 3). In some examples, the annular space 136 can be referred to as an inner lumen of the intermediate shaft 106. In some examples, the annular space 136 can be configured to receive an inflation fluid from a fluid source via the second port 140 of the adaptor 112 (for example, the annular space 136 can be in fluid communication with the second port 140 of the adaptor 112). The annular space 136 can be fluidly coupled to a fluid passageway 142 formed between the outer surface of the distal end portion of the inner shaft 108 and an inner surface of the balloon 118. As such, fluid from the fluid source can flow to the fluid passageway 142 from the annular space 136 to inflate the balloon 118 and radially expand and deploy the prosthetic valve.

In some examples, after crimping of the prosthetic valve onto the valve mounting portion 124, the distal tip portion 128 can be advanced over the proximal end portion 133 of the balloon 118. As a result, fluid arranged within the proximal end portion 133 of the balloon 118 can be displaced and pushed distally, within the balloon 118, to the distal end portion 132 of the balloon 118. The radially depressed, distal end portion 132 of the balloon 118 can then radially expand (for example, inflate partially) as it receives the displaced fluid to an expanded state. The radial depression 134 can be configured (for example, sized) so that the distal end portion 132 can receive the displaced fluid without radial expanding the portion of the balloon 118 within the valve mounting portion 124, thereby preventing the crimped profile of the prosthetic valve from increasing.

An inner lumen 144 of the inner shaft 108 can be configured to receive a guidewire therethrough, for navigating the distal end portion 109 of the delivery apparatus 100 to the target implantation site. As introduced above, the first port 138 of the adaptor 112 can be coupled to the inner lumen 144 and configured to receive the guidewire. For example, the distal end portion 109 of the delivery apparatus 100 can be advanced over the guidewire, to the target implantation site.

As shown in FIG. 2, the handle 102 can include a steering mechanism configured to adjust the curvature of the distal end portion 109 of the delivery apparatus 100. For example, the handle 102 can include an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion 109 of the delivery apparatus 100. Further details on steering or flex mechanisms for the delivery apparatus are described in U.S. Patent No. 9,339,384, which is incorporated by reference herein in its entirety.

The handle 102 can further include an adjustment mechanism 161 including an adjustment member, such as the illustrated rotatable knob 162. The adjustment mechanism 161 can be configured to move (thus adjust the axial position) of the intermediate shaft 106 relative to the outer shaft 104. The handle 102 can also include a locking mechanism configured to retain (for example, lock) the position of the intermediate shaft 106 relative to the handle 102. In some examples, the locking mechanism can include another adjustment member, which can be configured as a rotatable knob 178. In some examples, rotating the knob 178 to a locked position can cause the intermediate shaft 106 to frictionally engage with other components of the handle 102, thereby restraining movement of the intermediate shaft 106 for fine positioning of the prosthetic valve mounted on the distal end portion of the delivery apparatus 100. Rotating the knob 178 to an unlocked position allows axial and rotational movement of the intermediate shaft 106 relative to the proximal end portion of the handle 102. For example, rotation of the knob 162 can cause the intermediate shaft 106 to move axially relative to the outer shaft 104 (either in the proximal or distal direction, depending on the direction the knob 162 is rotated).

Further details on the adjustment mechanism and locking mechanism of the handle 102 can be found in U.S. Patent No. 9,339,384, which is incorporated by reference herein in its entirety. Additional examples of delivery apparatuses and related steering mechanism can be found in U.S. Patent No. 8,568,472, which is incorporated herein by reference in its entirety.

According to certain examples, to implant a prosthetic valve (for example, prosthetic valve 50) in a native heart valve of the patient, the delivery apparatus 100 can be introduced into vasculature of the patient. The prosthetic valve can be initially retained in a radially compressed configuration on the valve mounting portion 124 (and over the balloon 118) of the delivery apparatus 100.

In some instances, once inside the patient’s vasculature, the position (e.g., axial position) of the prosthetic valve relative to the balloon 118 can be adjusted such that the prosthetic valve 50 is centered on the balloon 118. In some instances, the axial position of the prosthetic valve relative to the balloon 118 may not be adjusted.

When navigating the prosthetic valve through an arched region of the vasculature (for example, the aortic arch), the curvature of the distal end portion 109 of the delivery apparatus 100 can be adjusted, for example, by rotating the knob 160 to increase or decrease the tension in the pull wire which extends between the handle 102 and the distal end of the outer shaft 104. The prosthetic valve can be positioned within or adjacent an annulus of the native heart valve. Prior to inflating the balloon 118, the outer shaft 104 can be retracted proximally away from the balloon 118 for a sufficient distance so that the outer shaft does not interfere with balloon inflation. This can be accomplished, for example, by holding the adaptor 112 stationary against the operating table and rotating the knob 162 in a direction that causes the handle 102 and the outer shaft 104 to move proximally away from the balloon 118. Then, the prosthetic valve can be radially expanded and deployed by inflating the balloon 118. Inflation of the balloon 118 can radially expand the prosthetic valve 50 so that the prosthetic valve 50 contacts the native annulus. The expanded prosthetic valve 50 becomes anchored within the native aortic annulus by the radial outward force of the valve’s frame against the surrounding tissue.

As described above, the knob 114 of the handle 102 can be configured to rotate the intermediate shaft 106, thereby rotating the balloon 118 mounted on the intermediate shaft 106 and a radially compressed prosthetic valve mounted on the balloon 118, around the valve mounting portion 124. Thus, rotating the knob 114 can rotate the prosthetic valve, around the central longitudinal axis 120, into a desired (circumferential or rotational) orientation relative to the native anatomy at the target implantation site.

For example, to implant a prosthetic valve (for example, prosthetic valve 50) at a native aortic valve, instead of deploying the prosthetic valve with the delivery apparatus 100 in a random rotational orientation relative to the aorta, which may result in commissures (for example, commissures 64) of the prosthetic valve being arranged in front of the coronary arteries, it may be desirable to deploy the prosthetic valve in a targeted rotational orientation where the commissures are positioned away from and do not block the coronary arteries (e.g., to reduce the likelihood blocking coronary access during subsequent interventional procedures). Specifically, the delivery apparatus 100 can be configured to control the rotational positioning of the prosthetic heart valve relative to the native valve, to achieve the commissure alignment, thereby increasing access to the coronary arteries. Additionally, this positioning of the prosthetic heart valve can facilitate a later, leaflet cutting procedure that provides increased access to the coronary arteries.

Further details on methods of aligning commissures of native valve with commissures of prosthetic valve after deploying the prosthetic valve are described in PCT Patent Publication No. WO 2022/046585, which is incorporated by reference herein in its entirety.

For mounting a prosthetic valve onto a valve mounting portion of a delivery apparatus (e.g., the delivery apparatus 100), in a specified axial position relative to the delivery apparatus, one or more positioning members can be used. The one or more positioning members can be configured such that they are perceptible to a user under visible light, and they are distinguishable from other parts of the delivery apparatus (such as the inner shaft 108 and balloon 118).

As used herein, visible light can be defined as the portion of the electromagnetic spectrum that is perceivable by a typical human eye in typical conditions (e.g., light with a wavelength within a range of 380-750 nanometers). Visible light is not ultraviolet nor x-rays (and thus, not fluoroscopy), which have a smaller wavelength than visible light, nor is it infrared, which has a larger wavelength than visible light. Thus, an object that is visible under visible light is visible to a human and may be visible under low visible light conditions (e.g., a dark or low light room).

In some examples, the positioning member can be an axially extending tube (cylinder), an axially extending strip, a ring, or the like that is disposed around a portion of the inner shaft, underneath the balloon.

In some examples, the positioning member has a distinguishing indicium visible to a user and distinguishable from the guidewire lumen (e.g., inner shaft 108), such as a distinguishing color, shape, texture, or the like. For example, in some instances, the positioning member can be various shapes (e.g., cylindrical, a strip, an oval, or the like), or comprise various shapes thereon (e.g., a shape on a tube or other base structure), such as multiple lines, an oval, a star, or the like.

In some instances, the distinguishing indicium can be a texture and/or raised form or shape that provides tactile feedback to a user (e.g., multiple raised lines, a stippling texture, a raised shape, or the like).

The positioning member is configured to indicate the desired location for an end of the prosthetic heart valve when mounted (and at least partially radially compressed or fully radially compressed) around the valve mounting portion of the delivery apparatus.

As introduced above, there may be an optimum, predetermined distance or offset from the distal shoulder 126 of the delivery apparatus for mounting the prosthetic valve onto the valve mounting portion 124 that results in more symmetric deployment of the prosthetic valve upon inflation of the balloon 118 at the implantation site. More symmetric deployment may refer to the prosthetic valve radially expanding with an hourglass or dog bone shape, or more symmetrical expansion diameter along its length, as the balloon inflates (as compared to asymmetric deployment where one end portion of the prosthetic valve expands more rapidly than the other end). Thus, the positioning member can indicate the predetermined distance from the distal shoulder 126 for placement of a distal end 192 of the prosthetic valve 190 on the valve mounting portion 124 (which can be any of the prosthetic valves described herein, such as the prosthetic valve 50, 300, or 400).

FIG. 3 and FIGS. 5A-5C show exemplary locations for a positioning member on the delivery apparatus 100. In some examples, a positioning member 150 can be disposed on and/or around the inner shaft 108, adjacent to and extending proximally (in a direction of the central longitudinal axis 120) from the distal shoulder 126. The positioning member 150 extends underneath the balloon 118, at a distal end portion of the valve mounting portion 124 of the delivery apparatus 100.

The positioning member 150 can be part of the distal shoulder 126, in some examples, as shown in FIGS. 3, 6, and 7. For example, the positioning member 150 can be integrated together with or formed (e.g., molded together) as one piece with the distal shoulder 126. A cross-sectional view of the distal shoulder 126, which includes the positioning member 150, is shown alone in FIG. 6 and a perspective view of an assembly 123 of the distal shoulder 126 coupled to the nose cone 122 is shown in FIG. 7. In some instances, the entire distal shoulder 126 shown in FIGS. 6 and 7, which includes the positioning member 150, can be injection molded.

The assembly 123, which includes the distal shoulder 126 (or valve retention member) and the nose cone 122 (or distal tip) can be referred to as a nose cone assembly 123 or distal tip assembly 123.

Thus, in some instances, the integrated part or single unitary component shown in FIG. 6 can be referred to as the distal shoulder 126 (or the valve retention member) which comprises the base portion 125, the flared portion 131 (which can comprise wings 130), and the positioning member 150 (or positioning member portion).

The positioning member 150 extends proximally from the base portion 125 of the distal shoulder 126 to a proximal end 152 of the positioning member 150. The proximal end 152 indicates a specified position (axial position) for the distal end 192 of the radially compressed (or at least partially radially compressed) prosthetic heart valve 190 when disposed around the valve mounting portion 124, as shown in FIGS. 5B and 5C.

For example, during pre-crimping of the prosthetic valve 190, the prosthetic valve 190 can be positioned around the valve mounting portion 124 and radially compressed most of the way (e.g., 90% of its fully crimped or radially compressed diameter) onto the valve mounting portion 124 with its distal end 192 aligned with (and in some instances, abutting) the proximal end 152 of the positioning member 150. In the partially crimped state, the prosthetic heart valve 190 is relatively easy to move along the valve mounting portion 124 of the delivery apparatus 100. Prior to fully crimping the prosthetic heart valve 190, the user can inspect the positioning of the prosthetic heart valve relative to the delivery apparatus to make sure that end of the prosthetic heart valve is aligned with (e.g., abutting or spaced less than 2 mm from) the positioning member 150. The prosthetic valve 190 can then be crimped to its final radially compressed diameter.

In some instances, the prosthetic valve 190 can be fully radially compressed and crimped onto the valve mounting portion 124 with its distal end 192 aligned with (e.g., abutting or spaced less than 2 mm from) the proximal end 152 of the positioning member 150.

A length of the positioning member 150 (a distance between its distal end where it extends proximally from the base portion 125 of the distal shoulder 126 and its proximal end 152) can be predetermined such that its proximal end 152 is offset by a set distance 154 from a proximal end 127 of the flared portion 131 or wings 130 of the distal shoulder 126, as depicted in FIG. 5B. As a result, the distal end 192 of the prosthetic valve 190 is spaced away (in the proximal direction) from the proximal end 127 of the flared portion 131 of the distal shoulder 126 by the set distance 154 (see, e.g., FIGS. 5B and 5C).

The set distance 154 can be predetermined based on the prosthetic valve (e.g., its size and/or configuration) such that the prosthetic valve radially expands more symmetrically upon deployment at the implantation site (e.g., via inflation of the balloon).

In some examples, as shown in FIG. 7, the positioning member 150 can be cylindrical such that it fully surrounds the inner shaft 108 and is visible to a user in any rotational orientation of the delivery apparatus. For example, in some instances, the positioning member 150 can have an annular cross-section (section that is normal to a central longitudinal axis 129 of the distal shoulder 126, as shown in FIG. 6).

In some examples, the positioning member 150 may not fully surround the inner shaft and can instead be an axially extending strip or partial tube (e.g., with a half-annular cross-section).

As introduced above, in some examples, the positioning member 150 can comprise a distinguishing indicium, such as a distinguishing color, shape, or texture. For example, in some instances, the distinguishing indicium or indicia can comprise a plurality of ridges, stippling, or another raised shape or texture that protrudes outward from a base structure (e.g., the tubular structure) and provides tactile feedback to a user.

In some instances, the distinguishing indicium can be a shape or symbol on the base structure of the positioning member.

In some examples, the positioning member can comprise more than one type of distinguishing indicium, such as a distinguishing color, a texture, and/or raised feature in various combinations.

As introduced above, the delivery apparatus 100 can include one or more radiopaque markers or marker bands 153 mounted on the inner shaft 108. In some examples, the positioning member 150 can be disposed at least partially around a first radiopaque marker band 153a. For example, the positioning member 150 can at least partially surround and axially overlap the first radiopaque marker band 153a. Said another way, the positioning member 150 can be disposed radially outside of the radiopaque marker band 153a and axially overlap at least a portion of the radiopaque marker band 153a.

In some instances, the radiopaque marker band 153a can be disposed within the positioning member 150, as depicted in FIG. 3. For illustration purposes, the marker band 153a is shown in dashed lines in FIGS. 5A-5C to denote its positioning underneath or within the positioning member 150.

Both the first radiopaque marker band 153a and the positioning member 150 can be configured to indicate a specified location for the distal end 192 of the radially compressed prosthetic valve 190 on the valve mounting portion 124.

However, since the radiopaque marker bands 153 can comprise a radiopaque material (e.g., a metal or metal alloy, such as platinum iridium), they may not be easily visible by a user (e.g., a medical technician) in a procedure room (which may be dimly lit or dark, in some instances). For example, the radiopaque marker bands 153 may not be easily distinguishable from the remainder of the delivery apparatus, including the inner shaft 108. Thus, the positioning member 150 can comprise a polymeric material in a highly visible color (and/or another distinguishing indicium, as described herein) relative to a remainder of the delivery apparatus. As a result, the positioning member 150 can be distinguishable while mounting the prosthetic valve 190 onto the valve mounting portion 124, and a user can easily align the prosthetic valve 190 with the positioning member 150, as described above.

During the implantation procedure, when the distal end portion of the delivery apparatus with the prosthetic valve mounted thereon is inside a patient’s body, the radiopaque marker bands 153 can be visible under medical imaging (e.g., fluoroscopy) to assist in positioning the prosthetic valve at the implantation site.

In this way, the positioning member 150 can be separate from and comprise a different material than the radiopaque marker bands 153. Further, while the positioning member 150 is used for axially positioning the prosthetic valve 190 onto the valve mounting portion 124 under visible light (even in a dark procedure room), the marker bands 153 are used for positioning the prosthetic valve 190 at the implantation site under fluoroscopy (or other medical imaging modalities).

As an example, the positioning member 150 can comprise a colored polymer. In some examples, the polymer of the positioning member 150 can be PEBAX. In some examples the polymer of the positioning member 150 can be a different polymer, such as polyurethane.

In some examples, the positioning member 150 can be a first color (for example, white, yellow, or a fluorescent color) that is visible under visible light, while the inner shaft 108 is a second color (for example, a darker color such as blue), the first color contrasting with the second color.

In some examples, the entire distal shoulder 126, including the positioning member 150, can be the same, contrasting first color.

In some examples, the entire assembly 123 can be the contrasting first color.

In some examples, the nose cone 122 can be a different color than the distal shoulder 126 (and the positioning member 150).

The first color of the positioning member 150 can be selected such that it is clearly visible to the human eye (without imaging) in a procedure room and underneath the formed balloon 118.

In some examples, the first color of the positioning member 150 (or any of the other positioning members described herein, such as positioning member 156), can be selected to match a color corresponding to a specifically sized prosthetic valve. For example, differently sized prosthetic valves (e.g., 23 mm, 26 mm, and 29 mm) can be used with the same delivery apparatus (the same type of delivery apparatus) but may have a different optimal axial position along the valve mounting portion, and thus the length and/or axial position of the positioning member 150 can be adjusted for differently sized valves to be mounted on the delivery apparatus.

For example, a first color (e.g., green) can correspond to a 23 mm prosthetic valve, and thus the positioning member 150 (or any of the other positioning members described herein, such as positioning member 156) can have the corresponding, first color, a second color (e.g., purple can correspond to a 26 mm prosthetic valve, and thus the positioning member can have the corresponding, second color, and a third color (e.g., orange) can correspond to a 29 mm prosthetic valve, and thus the positioning member can have the correspond, third color.

In some instances, a packaging for the prosthetic valve (e.g., for shipping and storage) and/or the prosthetic valve itself can be color-coded (e.g., green, purple, or orange) according to the valve’s size and match the coloring on the appropriately sized positioning member. For example, the prosthetic valve and/or packaging for the prosthetic valve can comprise a color indicator having a color corresponding to the valve size.

In some instances, the delivery apparatus 100 can include an indicator 163 (or color indicator) that has a color that matches the color of the positioning member and corresponds to the size of the prosthetic valve to be mounted on the delivery apparatus. Though the indicator 163 is shown on the distal end of the handle 102 in FIG. 2, in some instances, the indicator 163 can be on a different portion of the handle 102 or other component of the delivery apparatus. As a result, a user can more easily select and verify the correctly sized prosthetic valve to be mounted on the delivery apparatus for implantation (e.g., by matching the color of the valve packaging and/or valve to the color of the positioning member and indicator 163).

By integrating the positioning member 150 with the distal shoulder 126, such that a single part is formed, manufacturing of the combined distal shoulder 126 (with positioning member 150) can be simplified and assembly of the delivery apparatus can be made easier (e.g., without additional steps for assembling a separate positioning member 150 to the inner shaft 108).

However, in some examples, the positioning member 150 can be a separate component from the distal shoulder 126 that is assembled onto the inner shaft 108 at the specified axial position (e.g., as shown in FIGS. 5A-5C). FIG. 8 shows an exemplary positioning member 250 that is formed as a separate component that can be assembled onto the inner shaft 108.

For example, the positioning member 250 can be a tube or partial tube (e.g., colored tube or tubing) that extends proximally from the distal shoulder 126 to the specified axial location on the inner shaft 108 such that its proximal end 252 is offset by the set distance 154 from the proximal end of the distal shoulder 126.

In some instances, a distal end 253 of the positioning member 250 can abut the base portion 125 of the distal shoulder 126 (FIG. 8).

In some examples, during assembly of the delivery apparatus, the positioning member 250 can be mounted over the inner shaft 108, in the specified axial position, and the distal shoulder 126 can be mounted onto the inner shaft (with the positioning member 250 abutting the distal shoulder 126, in some instances).

As shown in FIG. 5C, in some examples, another positioning member 156 can be disposed around the inner shaft 108 at a proximal end of the valve mounting portion 124. For example, in some instances, the positioning member 156 can be a second positioning member that is formed as a cylinder or tubing and arranged around (and/or mounted to) the inner shaft 108. The positioning member 156 can be disposed within the proximal end portion 133 of the balloon 118.

In the instances where the delivery apparatus includes both the first positioning member 150 and the second positioning member 156, an axial distance or gap between the first and second positioning members 150, 156 can match a length of the radially compressed or crimped prosthetic valve 190 (as shown in FIG. 5C). This distance or gap between positioning members can be adjusted for differently sized valves. For example, for a 29 mm prosthetic valve, the crimped length of the valve and distance between the two positioning members can be 29 mm ± 1mm, whereas for a 23 mm prosthetic valve the crimped length of the valve and distance between the two positioning members can be 23 mm ± 1mm.

In some instances, the position member 156 can be the sole positioning member and the positioning member 150 can be omitted.

A distal end 158 of the positioning member 156 can indicate a specified position (axial position) for the proximal end 194 of the radially compressed (or at least partially radially compressed) prosthetic heart valve 190 when disposed around the valve mounting portion 124.

In some examples, the proximal end 194 of the prosthetic valve 190 can abut the distal end 158 of the positioning member 156.

The positioning member 156 can be configured similarly to the positioning member 150 (e.g., comprising a colored polymeric material that contrasts in color with the inner shaft 108 and is identifiable by a user under visible light).

In some examples, the positioning member 156 is arranged around a second radiopaque marker band 153b. For illustration purposes, the marker band 153b is shown in dashed lines in FIG. 5C to denote its positioning underneath or within the positioning member 156.

In this way, by utilizing one or more highly visible positioning members on a valve mounting portion of a delivery apparatus, a prosthetic valve can be easily and efficiently mounted around the valve mounting portion in a specified axial position along the valve mounting portion. For example, by constructing the one or more positioning members out of a colored polymer (or another distinguishing indicium or indicia) that contrasts from a remainder of the delivery apparatus (or at least the inner shaft or guidewire lumen) and that is easily identifiable by the naked eye in a procedure room (which may be dark or dimly lit), the prosthetic valve can be more easily and accurately positioned on the valve mounting portion by a user, prior to an implantation procedure. As a result of mounting the prosthetic valve in the specified axial position, during expansion of the prosthetic valve at an implantation site (e.g., by inflating a balloon of the delivery apparatus), the prosthetic valve can radially expand more symmetrically along its length (from inflow to outflow end), thereby enabling the prosthetic valve to be implanted at the native anatomy more accurately and securely.

In some examples, the positioning member can be integrated, or formed as one piece, with a portion of the distal end portion of the delivery apparatus, such as the distal shoulder (or distal shoulder/nose cone assembly). This can simplify manufacturing and assembly of the delivery apparatus.

The positioning members described above can be adapted (e.g., sized) for use with various delivery apparatuses and/or prosthetic valves. For example, the delivery apparatus 100 and/or a similar delivery apparatus can be configured to receive and deliver a variety of prosthetic valves (or a variety of prosthetic devices, such as stents) to an implantation site. The positioning device, such as its length, amount it extends proximally from the distal shoulder or another polymeric body mounted on a distal end portion of a delivery apparatus, or is axial positioning along the distal end portion of the delivery apparatus, can be predetermined based on the type and/or size of the prosthetic device to be mounted on a mounting portion of the delivery apparatus.

FIGS. 9 and 10 show examples of prosthetic valves that can be mounted to a delivery apparatus (e.g., the delivery apparatus 100 or a similar delivery apparatus) and positioned on a valve mounting portion of the delivery apparatus with one or more of the positioning members described herein.

For example, FIG. 9 shows a prosthetic heart valve 300 (e.g., prosthetic valve) configured as a balloon expandable prosthetic heart valve. The prosthetic valve 300 has four main components: a stent or frame 312, a valvular structure 314, an inner skirt 316, and a perivalvular outer sealing member or outer skirt 318. The prosthetic valve 300 has an inflow end portion 315, an intermediate portion 317, and an outflow end portion 319.

The valvular structure 314 can comprise three leaflets 340, collectively forming a leaflet structure, which can be arranged to collapse in a tricuspid arrangement, although in other embodiments there can be greater or fewer number of leaflets (e.g., one or more leaflets 340). The leaflets 340 can be secured to one another at their adjacent sides to form commissures 322 of the valvular (e.g., leaflet) structure 314. The lower edge of valvular structure 314 can have an undulating, curved scalloped shape and can be secured to the inner skirt 316 by sutures (not shown).

The frame 312 can be formed with a plurality of circumferentially spaced slots, or commissure windows 320 that are adapted to mount the commissures 322 of the valvular structure 314 to the frame. The frame 312 can be made of any of various suitable plastically-expandable materials or self-expanding materials, as described herein. Additional details regarding the prosthetic valve 300 and its various components are described in PCT Publication No. WO 2018/222799, which is incorporated herein by reference.

FIG. 10 shows a prosthetic valve 400 comprising a radially expandable and/or compressible annular frame 402, a plurality of leaflets 404 mounted within the frame 402, and an outer skirt 406 secured to and around an outer surface of the frame 402. The frame 402 can comprises a plurality of interconnected struts 414 and a plurality of apices 408 that are spaced circumferentially apart around an inflow end portion 416 and an outflow end portion 418 of the frame 402 (only the apices 408 at the outflow end portion 418 are visible in FIG. 10). Each apex 408 is formed at a junction between two angled struts 414 at either the inflow end portion 416 or the outflow end portion 418. The frame 402 comprises a plurality of axially extending posts 410, some of which define commissure windows therein. Commissure tabs of adjacent leaflets 404 can be paired together and extend through the commissure windows, thereby forming commissures 412 secured to the frame 402 which protrude radially outward from the frame 402. In some examples, the prosthetic valve 400 can be a balloon expandable valve. Additional details on the prosthetic valve 400 can be found in PCT Publication No. WO/2022/226147, which is incorporated by reference herein.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient’s vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example1. A delivery apparatus comprising: a shaft defining a lumen for a guidewire, wherein the shaft is a first color; an inflatable balloon overlaying a distal end portion of the shaft, wherein the balloon is configured to receive a prosthetic valve in a radially compressed state; a radiopaque marker mounted to the distal end portion of the shaft; and a polymeric positioning member disposed around the distal end portion of the shaft and inside the balloon, wherein the positioning member is a second color, and wherein the positioning member is configured to indicate an axial position for an end of the prosthetic valve when the prosthetic valve is mounted around the balloon.

Example 2. The delivery apparatus of any example herein, particularly example 1, wherein the second color contrasts with the first color and is distinguishable under visible light.

Example 3. The delivery apparatus of any example herein, particularly either example 1 or example 2, wherein the radiopaque marker comprises platinum iridium.

Example 4. The delivery apparatus of any example herein, particularly any one of examples 1-3, wherein the positioning member is positioned around the radiopaque marker.

Example 5. The delivery apparatus of any example herein, particularly any one of examples 1-4, wherein the positioning member is an axially extending tube.

Example 6. The delivery apparatus of any example herein, particularly any one of examples 1-5, wherein the positioning member comprises a texture.

Example 7. The delivery apparatus of any example herein, particularly any one of examples 1-6, wherein the positioning member is disposed within a distal end portion of the balloon and indicates the axial position for a distal end of the prosthetic valve when the prosthetic valve is mounted around the balloon.

Example 8. The delivery apparatus of any example herein, particularly example 7, further comprising a distal shoulder mounted to the distal end portion of the shaft and disposed within the distal end portion of the balloon, wherein the distal shoulder is configured to resist axial movement of the prosthetic valve, in a distal direction relative to the balloon, when the prosthetic valve is mounted around the balloon, and wherein the positioning member extends in a proximal direction from the distal shoulder.

Example 9. The delivery apparatus of any example herein, particularly example 8, wherein the positioning member is molded as one piece with the distal shoulder, and wherein the positioning member extends past and proximal to a proximal end of a flared portion of the distal shoulder.

Example 10. The delivery apparatus of any example herein, particularly example 9, wherein the flared portion comprises a plurality of wings that extend radially outward, in the proximal direction, from a base portion of the distal shoulder.

Example 11. The delivery apparatus of any example herein, particularly example 10, wherein the base portion is coupled to a nose cone which forms a distal tip of the delivery apparatus.

Example 12. The delivery apparatus of any example herein, particularly any one of examples 7-11, wherein the positioning member is a first positioning member, and further comprising a second positioning member disposed around the shaft and within a proximal end portion of the balloon, and wherein the second positioning member indicates the axial position for a proximal end of the prosthetic valve when the prosthetic valve is mounted around the balloon.

Example 13. The delivery apparatus of any example herein, particularly any one of examples 1-12, wherein the shaft is an inner shaft of the delivery apparatus, and further comprising a handle and an outer shaft extending distally from the handle, wherein the inner shaft extends through the outer shaft.

Example 14. The delivery apparatus of any example herein, particularly example 13, wherein the outer shaft is steerable, and wherein the handle includes a steering mechanism configured to adjust a curvature of a distal end portion of the outer shaft.

Example 15. The delivery apparatus of any example herein, particularly either example 13 or example 14, further comprising an intermediate shaft extending through the outer shaft, wherein the inner shaft extends through the intermediate shaft and distal to a distal end of the intermediate shaft, and wherein a proximal end of the balloon is coupled to the distal end of the intermediate shaft.

Example 16. An assembly comprising the delivery apparatus of any example herein, particularly any one of examples 1-15, and the prosthetic valve, wherein the prosthetic valve is radially collapsible to a collapsed configuration and radially expandable to an expanded configuration, the prosthetic valve mounted around the balloon in the collapsed configured with its distal end axially aligned with a proximal end of the positioning member.

Example 17. A delivery apparatus comprising: a shaft defining a lumen for a guidewire; an inflatable balloon arranged around a distal end portion of the shaft and overlaying a valve mounting portion of the delivery apparatus that is configured to receive a prosthetic valve in a radially compressed state; and a distal shoulder mounted on the distal end portion of the shaft and arranged within a distal end portion of the balloon, wherein the distal shoulder comprises a base portion, a flared portion that extends radially outward from the base portion and proximally toward the valve mounting portion, and a positioning member extending axially from the base portion and proximal to a proximal end of the flared portion, wherein the positioning member is formed as one piece with a remainder of the distal shoulder.

Example 18. The delivery apparatus of any example herein, particularly example 17, wherein the proximal end of the flared portion is arranged adjacent to the valve mounting portion, and wherein the positioning member extends over the shaft.

Example 19. The delivery apparatus of any example herein, particularly example 18, wherein the positioning member fully surrounds a portion of the shaft.

Example 20. The delivery apparatus of any example herein, particularly any one of examples 17-19, wherein the flared portion comprises a plurality of circumferentially spaced apart wings that flare radially outward from the base portion toward the valve mounting portion.

Example 21. The delivery apparatus of any example herein, particularly any one of examples 17-20, further comprising a nose cone coupled to a distal end of the distal shoulder, wherein the nose cone forms a distal tip of the delivery apparatus.

Example 22. The delivery apparatus of any example herein, particularly any one of examples 17-21, wherein the distal shoulder comprises a polymer.

Example 23. The delivery apparatus of any example herein, particularly any one of examples 17-22, wherein the distal shoulder is a first color, and wherein the shaft is a second color, the first color distinguishable from the second color under visible light.

Example 24. The delivery apparatus of any example herein, particularly any one of examples 17-23, further comprising a radiopaque marker band mounted around the distal end portion of the shaft, and wherein the positioning member covers at least a portion of the radiopaque marker band.

Example 25. The delivery apparatus of any example herein, particularly example 24, wherein the radiopaque marker band is disposed within the positioning member.

Example 26. The delivery apparatus of any example herein, particularly either example 24 or example 25, wherein the radiopaque maker band comprises platinum iridium, and wherein the positioning member comprises a colored polymer that is distinguishable from the shaft under visible light.

Example 27. The delivery apparatus of any example herein, particularly any one of examples 17-26, wherein the positioning member is a distal positioning member and is configured such that a distal end of the prosthetic valve abuts the distal positioning member when the prosthetic valve is mounted around the valve mounting portion and balloon.

Example 28. The delivery apparatus of any example herein, particularly example 27, further comprising a proximal positioning member disposed around the shaft adjacent to a proximal end portion of the valve mounting portion, wherein the proximal positioning member is configured such that a proximal end of the prosthetic valve abuts the proximal positioning member when the prosthetic valve is mounted around the valve mounting portion and balloon.

Example 29. The delivery apparatus of any example herein, particularly any one of examples 17-28, wherein a proximal end of the positioning member is configured to indicate an axial position for a distal end of the prosthetic valve when the prosthetic valve is mounted around the balloon on the valve mounting portion of the delivery apparatus.

Example 30. The delivery apparatus of any example herein, particularly any one of examples 17-29, wherein the shaft is an inner shaft of the delivery apparatus, and further comprising a handle and an outer shaft extending distally from the handle, wherein the inner shaft extends through the outer shaft.

Example 31. The delivery apparatus of any example herein, particularly example 30 wherein the outer shaft is steerable, and wherein the handle includes a steering mechanism configured to adjust a curvature of a distal end portion of the outer shaft.

Example 32. A valve retention member for a delivery apparatus for a prosthetic valve, comprising: a base portion configured to be mounted to a distal end portion of a shaft of the delivery apparatus; a flared portion that extends radially outward and proximally from the base portion, wherein a proximal direction is parallel to a central longitudinal axis of the valve retention member; and a positioning member that extends proximally from the base portion and past a proximal end of the flared portion, wherein the positioning member has a distinguishing indicium visible to a user to aid in positioning of the prosthetic valve onto the delivery apparatus during crimping of the prosthetic valve to the delivery apparatus, and wherein the positioning member, flared portion, and base portion are formed together as a unitary component.

Example 33. The valve retention member of any example herein, particularly example 32, wherein a proximal end of the positioning member is offset from the proximal end of the flared portion, in the proximal direction, by a predetermined distance.

Example 34. The valve retention member of any example herein, particularly either example 32 or example 33, wherein the positioning member, flared portion, and base portion are injection molded together.

Example 35. The valve retention member of any example herein, particularly any one of examples 32-34, wherein the valve retention member comprises a polymer.

Example 36. The valve retention member of any example herein, particularly any one of examples 32-35, wherein the indicium of the positioning member is a first color that is distinguishable to the user under visible light.

Example 37. The valve retention member of any example herein, particularly any one of examples 32-35, wherein the indicium of the positioning member is a texture or raised feature.

Example 38. The valve retention member of any example herein, particularly example 37, wherein the texture or raised feature is a plurality of ridges.

Example 39. The valve retention member of any example herein, particularly any one of examples 32-35, wherein the indicium of the positioning member is a visible shape or symbol on a base structure of the positioning member.

Example 40. The valve retention member of any example herein, particularly any one of examples 32-39, wherein the flared portion comprises a plurality of spaced apart wings that flare radially outward from the base portion.

Example 41. The valve retention member of any example herein, particularly any one of examples 32-40, wherein the positioning member has an annular cross-section.

Example 42. A nose cone assembly comprising the valve retention member of any example herein, particularly any one of examples 32-41 and further comprising a nose cone coupled to a distal end of the base portion of the retention member, wherein the nose cone forms a distal tip of the delivery apparatus.

Example 43. A delivery apparatus comprising: a shaft defining a lumen for a guidewire; an inflatable balloon arranged around a distal end portion of the shaft and overlaying a valve mounting portion of the delivery apparatus that is configured to receive a prosthetic valve in a radially compressed state; and the valve retention member of any one of claims 32-41, wherein the valve retention member is mounted on the distal end portion of the shaft and arranged within the balloon.

Example 44. The delivery apparatus of any example herein, particularly example 43, wherein the positioning member is a first color, and the shaft is a second color, the first color distinguishable from the second color.

Example 45. The delivery apparatus of any example herein, particularly example 43, wherein the valve retention member is a first color, and the shaft is a second color, the first color distinguishable from the second color.

Example 46. The delivery apparatus of any example herein, particularly any one of examples 43-45, further comprising a radiopaque marker mounted to the distal end portion of the shaft, and wherein the positioning member is disposed radially outside of the radiopaque marker and axially overlaps at least a portion of the radiopaque marker.

Example 47. The delivery apparatus of any example herein, particularly example 46, wherein the radiopaque marker comprises platinum iridium.

Example 48. The delivery apparatus of any example herein, particularly any one of examples 43-47, wherein the shaft is an inner shaft of the delivery apparatus, and further comprising a handle and an outer shaft extending distally from the handle, wherein the inner shaft extends through the outer shaft and distal to a distal end of the outer shaft.

Example 49. The delivery apparatus of any example herein, particularly example 48, wherein the outer shaft is steerable, and wherein the handle includes a steering mechanism configured to adjust a curvature of a distal end portion of the outer shaft.

Example 50. The delivery apparatus of any example herein, particularly either example 48 or example 49, further comprising an intermediate shaft extending through the outer shaft, wherein the inner shaft extends through the intermediate shaft and distal to a distal end of the intermediate shaft, and wherein a proximal end of the balloon is coupled to the distal end of the intermediate shaft.

Example 51. The delivery apparatus of any example herein, particularly example 50, wherein a distal end of the balloon is coupled to a distal end of the valve retention member.

Example 52. The delivery apparatus of any example herein, particularly any one of examples 43-51, wherein the positioning member of the valve retention member is a first positioning member, and further comprising a second positioning member mounted on the shaft and spaced axially away from and proximal to the first positioning member, wherein the second positioning member has a distinguishing indicium visible to a user to aid in positioning of the prosthetic valve onto the delivery apparatus during crimping of the prosthetic valve to the delivery apparatus.

Example 53. An assembly comprising: the delivery apparatus of any example herein, particularly any one of examples 43-52; and a prosthetic valve that is radially collapsible to a collapsed configuration and radially expandable to an expanded configuration, the prosthetic valve mounted around the balloon on the valve mounting portion of the delivery apparatus in the collapsed configured with its distal end axially aligned with a proximal end of the positioning member.

Example 54. The assembly of any example herein, particularly example 53, wherein the distal end of the prosthetic valve abuts the proximal end of the positioning member.

Example 55. The assembly of any example herein, particularly either example 53 or example 54, further comprising a proximal positioning member disposed around the shaft adjacent to a proximal end portion of the valve mounting portion, wherein a proximal end of the prosthetic valve abuts a distal end of the proximal positioning member.

Example 56. The assembly of any example herein, particularly any one of examples 53-55, wherein the positioning member is a first color and corresponds to a color indicator on a packaging of the prosthetic valve.

Example 57. The assembly of any example herein, particularly any one of examples 53-56, wherein the positioning member is a first color and corresponds to a color of a color indicator on the prosthetic valve.

Example 58. The assembly of any example herein, particularly any one of examples 53-57, wherein the positioning member is a first color and corresponds to a color of a color indicator on the delivery apparatus.

Example 59. The assembly of any example herein, particularly example 58, wherein the delivery apparatus comprises a handle, the shaft extending distally from the handle, and wherein the color indicator is disposed on the handle.

Example 60. A method comprising sterilizing the prosthetic heart valve, apparatus, and/or assembly of any example.

Example 61. A prosthetic heart valve of any one of examples 1-59, wherein the prosthetic heart valve is sterilized.

The features described herein with regard to any example can be combined with other features described in any one or more of the other examples, unless otherwise stated. For example, any one or more of the features of one delivery apparatus can be combined with any one or more features of another delivery apparatus. As another example, any one or more features of one valve retention member can be combined with any one or more features of another valve retention member.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A delivery apparatus comprising:
a shaft defining a lumen for a guidewire, wherein the shaft is a first color;
an inflatable balloon overlaying a distal end portion of the shaft, wherein the balloon is configured to receive a prosthetic valve in a radially compressed state;
a radiopaque marker mounted to the distal end portion of the shaft; and
a polymeric positioning member disposed around the distal end portion of the shaft and inside the balloon, wherein the positioning member is a second color, and wherein the positioning member is configured to indicate an axial position for an end of the prosthetic valve when the prosthetic valve is mounted around the balloon.

2. The delivery apparatus of claim 1, wherein the second color contrasts with the first color and is distinguishable under visible light.

3. The delivery apparatus of claim 1, wherein the radiopaque marker comprises platinum iridium.

4. The delivery apparatus of claim 1, wherein the positioning member is positioned around the radiopaque marker.

5. The delivery apparatus of claim 1, wherein the positioning member is an axially extending tube.

6. The delivery apparatus of claim 1, wherein the positioning member comprises a texture.

7. The delivery apparatus of claim 1, wherein the positioning member is disposed within a distal end portion of the balloon and indicates the axial position for a distal end of the prosthetic valve when the prosthetic valve is mounted around the balloon.

8. The delivery apparatus of claim 7, further comprising a distal shoulder mounted to the distal end portion of the shaft and disposed within the distal end portion of the balloon, wherein the distal shoulder is configured to resist axial movement of the prosthetic valve, in a distal direction relative to the balloon, when the prosthetic valve is mounted around the balloon, and wherein the positioning member extends in a proximal direction from the distal shoulder.

9. The delivery apparatus of claim 8, wherein the positioning member is molded as one piece with the distal shoulder, and wherein the positioning member extends past and proximal to a proximal end of a flared portion of the distal shoulder.

10. The delivery apparatus of claim 9, wherein the flared portion comprises a plurality of wings that extend radially outward, in the proximal direction, from a base portion of the distal shoulder.

11. The delivery apparatus of claim 10, wherein the base portion is coupled to a nose cone which forms a distal tip of the delivery apparatus.

12. The delivery apparatus of claim 7, wherein the positioning member is a first positioning member, and further comprising a second positioning member disposed around the shaft and within a proximal end portion of the balloon, and wherein the second positioning member indicates the axial position for a proximal end of the prosthetic valve when the prosthetic valve is mounted around the balloon.

13. An assembly comprising the delivery apparatus of claim 1, and the prosthetic valve, wherein the prosthetic valve is radially collapsible to a collapsed configuration and radially expandable to an expanded configuration, the prosthetic valve mounted around the balloon in the collapsed configured with its distal end axially aligned with a proximal end of the positioning member.

14. A valve retention member for delivery apparatus for a prosthetic valve, comprising:
a base portion configured to be mounted to a distal end portion of a shaft of the delivery apparatus;
a flared portion that extends radially outward and proximally from the base portion, wherein a proximal direction is parallel to a central longitudinal axis of the valve retention member; and
a positioning member that extends proximally from the base portion and past a proximal end of the flared portion, wherein the positioning member has distinguishing indicium visible to a user to aid in positioning of the prosthetic valve onto the delivery apparatus during crimping of the prosthetic valve to the delivery apparatus, and wherein the positioning member, flared portion, and base portion are formed together as a unitary component.

15. The valve retention member of claim 14, wherein a proximal end of the positioning member is offset from the proximal end of the flared portion, in the proximal direction, by a predetermined distance.

16. The valve retention member of claim 14, wherein the indicium of the positioning member is a first color that is distinguishable to the user under visible light.

17. The valve retention member of claim 14, wherein the indicium of the positioning member is a texture or raised feature.

18. The valve retention member of claim 14, wherein the indicium of the positioning member is a visible shape or symbol on a base structure of the positioning member.

19. The valve retention member of claim 14, wherein the positioning member has an annular cross-section.

20. A delivery apparatus comprising:
a shaft defining a lumen for a guidewire;
an inflatable balloon arranged around a distal end portion of the shaft and overlaying a valve mounting portion of the delivery apparatus that is configured to receive a prosthetic valve in a radially compressed state; and
the valve retention member of claim 14, wherein the valve retention member is mounted on the distal end portion of the shaft and arranged within the balloon.
